(19) United States
(12) Patent Application Publication
(10) Pub. No.: US 2026/0338790 A1
(43) Pub. Date: Oct. 29, 2026

(54) METHODS FOR IDENTIFYING AND CORRECTING TUMOR HUMORAL IMMUNE DYSFUNCTION

(71) Applicant: The Regents of the University of California, Oakland, CA (US)

(72) Inventor: Aaron S. MEYER, Los Angeles, CA (US)

(73) Assignee: The Regents of the University of California, Oakland, CA (US)

(21) Appl. No.: 19/491,103

(22) PCT Filed: Jun. 7, 2024
(86) PCT No.: PCT/US2024/032940

§ 371 Date: Dec. 8, 2025

Related U.S. Application Data
(60) Provisional application No. 63/472,099, filed on Jun. 9, 2023.

(51) Int. Cl. G01N 33/68; G01N 33/575

(52) U.S. Cl. CPC G01N 33/6854; G01N 33/575; G01N 2800/52

(57) ABSTRACT

Methods are provided for detecting and correcting defective anti-tumor antibody responses by identifying Fc properties therein, enhancing such properties, and administering enhanced anti-tumor antibodies to a subject, or administering enzymes that enhance Fc properties in situ.

### CROSS-REFERENCE TO RELATED APPLICATION

This application claims priority to U.S. Provisional Patent Application Ser. No. 63/472,099, filed Jun. 9, 2023, which is incorporated herein by reference in its entirety.

### BACKGROUND

Decades of foundational work has revealed that antibody (Ab) responses are widespread and abundant against many tumor types. Anti-tumor antibodies (ATAbs) display features of an active humoral immune response, including affinity enrichment through somatic hypermutation and high tumor selectivity. However, despite all the necessary components for eliminating cancer cells, ATAbs only weakly induce tumor cell killing in vitro and are ineffective at or prevent tumor elimination in vivo.

It is toward identifying defects in the anti-tumor antibody response and correcting it, that the present disclosure is directed.

### SUMMARY

In one aspect, a method is provided for identifying an effective therapeutic regimen for a subject having a tumor and endogenous anti-tumor antibodies, the method comprising the steps of:
a. obtaining a sample of the tumor and/or a bodily fluid from the subject;
b. isolating antibodies bound to the tumor and/or antibodies in the bodily fluid that bind to the tumor;
c. identifying and quantifying antibody types among the isolated antibodies;
d. identifying and quantifying IgG antibody subtypes among any IgG antibodies therein; and
e. identifying among each of the antibody types and/or subtypes therein one or more Fc properties comprising from among:
   1. extent of Fc sialylation, wherein increased Fc sialylation indicates defective IgG effector function or active suppression of IgG effector function; and/or
   2. extent of Fc fucosylation, wherein increased Fc fucosylation indicates defective IgG effector function; and/or
   3. extent of FcγRIIa interaction, wherein decreased FcγRIIa interaction indicates defective IgG effector function; and/or
   4. extent of FcγRIIIa interaction, wherein decreased FcγRIIIa interaction indicates defective IgG effector function; and/or
   5. extent of FcγRIIb interaction, wherein increased FcγRIIb interaction indicates defective IgG effector function; and/or
   6. extent of FcγRIV interaction, wherein decreased FcγRIV interaction indicates defective IgG effector function; and/or
   7. extent of IgG4, wherein high levels of enrichment in IgG4 indicates a deficient anti-tumor antibody response; and/or
   8. extent of IgG2, wherein high levels of or enrichment in IgG2 indicates a deficient anti-tumor antibody response; and
wherein:
   x. a defective antibody Fc effector function indicates a therapeutic regimen comprising administering endogenous anti-tumor antibodies after antibody modification thereof; and/or
y. a defective antibody Fc effector function indicates a therapeutic regimen comprising administering endogenous anti-tumor antibodies after depletion of antibodies with defective effector function; and/or
z. a defective antibody Fc effector function indicates a therapeutic regimen comprising administering endogenous anti-tumor antibodies after enrichment of antibodies with active effector function; and/or
aa. a defective antibody Fc effector function indicates a therapeutic regimen comprising administering an enzyme, such as a desialylase, e.g., neuraminidase, or a defucosylase, e.g., alpha-fucosidase, to modify endogenous anti-tumor antibodies in situ, localized to the tumor; and/or
bb. a deficient anti-tumor antibody response indicates a therapeutic regimen comprising depletion of IgG4 and/or enrichment in IgG3; and/or
cc. a deficient anti-tumor antibody response indicates a therapeutic regimen comprising depletion of IgG2 and/or IgG4, and/or enrichment in IgG1 and/or IgG3 over IgG2 and/or IgG4.

In some embodiments, the endogenous anti-tumor antibodies weakly induce tumor cell killing in vitro and/or are ineffective at or prevent tumor elimination in vivo. In some embodiments, the endogenous anti-tumor antibodies have defective Fc effector function or suppress IgG effector function.

In some embodiments, the extent of Fc sialylation is determined by Siglec-7 binding. In some embodiments, the extent of Fc sialylation is determined by Siglec-3 binding. In some embodiments, the extent of Fc sialylation is determined by Sambucus nigra agglutinin (SNA) binding. In some embodiments, the extent of Fc fucosylation is determined by FcγRIIa and FcγRIIIa interaction. In some embodiments, the extent of Fc fucosylation is determined by FcγRIIa interaction. In some embodiments, the extent of Fc fucosylation is determined by FcγRIIIa interaction. In some embodiments, the extent of FcγRIIIa interaction is determined by receptor binding. In some embodiments, the extent of Fc fucosylation is determined by the ratio of FcγRIIIa to FcγRIIa interaction. In some embodiments, the extent of FcγRIIa interaction is determined by receptor binding. In some embodiments, the extent of FcγRIIb interaction is determined by receptor binding. In some embodiments, the extent of FcγRIV interaction is determined by receptor binding.

In some embodiments, the content of IgG4 and ratio to IgG3 is determined.

In some embodiments, the content of IgG2 and ratio to IgG1 is determined.

In some embodiments, increased Fc sialylation indicates defective IgG effector function or active suppression of IgG effector function. In some embodiments, increased Fc fucosylation indicates defective IgG effector function or active suppression of IgG effector function. In some embodiments, decreased interaction with FcγRIIIa, FcγRIIa, FcγRIV, or any combination thereof indicates defective IgG effector function. In some embodiments, increased interaction with FcγRIIb indicates defective IgG effector function. In some embodiments, enriched IgG4 indicates defective anti-tumor antibody effector function. In some embodiments, high IgG4 content or ratio to IgG1 and/or IgG3 indicates a deficient anti-tumor antibody response. In some embodiments, enriched IgG2 indicates defective anti-tumor antibody effector function. In some embodiments, high IgG2 content or ratio to IgGI and/or IgG3 indicates a deficient anti-tumor antibody response.

In some embodiments, the tumor binding property of each antibody type and/or subtype is determined. In some embodiments, the method further comprises identifying and/or quantifying among the isolated antibodies any such antibodies that recognize specific tumor markers on the tumor with the properties above delineated for each marker. In some embodiments, the effector function attributes are determined before, during and/or after tumor therapy. In some embodiments, tumor therapy comprises surgery, radiation, immunotherapy, chemotherapy, or any combination thereof. In some embodiments, effector functional attributes are among cell killing, phagocytosis, complement response, cytokine release, and response to vaccination. In some embodiments, the bodily fluid is blood, plasma, serum, ascites or pleural effusion.

In some embodiments, antibody modification comprises correcting defective antibody glycosylation. In some embodiments, correcting defective antibody glycosylation comprises desialylating the antibodies, such as by using a neuraminidase. In some embodiments, correcting defective antibody glycosylation comprises defucosylating the antibodies, such as by using alpha-fucosidase. In some embodiments, correcting defective Fc effector function comprises depleting IgG4. In some embodiments, correcting defective Fc effector function comprises depleting IgG2.

In one aspect, a method is provided for treating a subject having a tumor and endogenous anti-tumor antibodies comprising identifying the effective therapeutic regimen as described herein above, comprising modifying the endogenous anti-tumor antibodies to enhance anti-tumor activity, and administering the modified antibodies to the subject. In some embodiments, the modified anti-tumor antibodies are administered parenterally or intratumorally. In some embodiments, enzymes to correct defective antibody glycosylation are administered in vivo or in situ.

In one aspect, a method is provided for enhancing the anti-tumor efficacy of endogenous anti-tumor antibodies from a subject having a tumor and endogenous anti-tumor antibodies, the method comprising the steps of:
a. obtaining a sample of the tumor and/or a bodily fluid from the subject;
b. isolating antibodies bound to the tumor and/or antibodies in the bodily fluid that bind to the tumor;
c. identifying and quantifying antibody types among the isolated antibodies;
d. identifying and quantifying IgG antibody subtypes among any IgG antibodies therein; and
e. identifying among each of the antibody types and/or subtypes therein one or more Fc properties comprising from among:
   1. extent of Fc sialylation, wherein increased Fc sialylation indicates defective IgG effector function or active suppression of IgG effector function; and/or
   2. extent of Fc fucosylation, wherein increased Fc fucosylation indicates defective IgG effector; and/or
   3. extent of FcγRIIa interaction, wherein decreased FcγRIIa interaction indicates defective IgG effector function; and/or
   4. extent of FcγRIIIa interaction, wherein decreased FcγRIIIa interaction indicates defective IgG effector function; and/or
   5. extent of FcγRIIb interaction, wherein increased FcγRIIb interaction indicates defective IgG effector function; and/or
   6. extent of FcγRIV interaction, wherein decreased FcγRIV interaction indicates defective IgG effector function; and/or
   7. extent of IgG4, wherein high levels of or enrichment in IgG4 indicates a deficient anti-tumor antibody response; and/or
   8. extent of IgG2, wherein high levels of or enrichment in IgG2 indicates a deficient anti-tumor antibody response; and
f. optionally identifying and quantifying among the isolated antibodies any such antibodies that recognize specific tumor markers on the tumor with the properties above delineated for each marker;
wherein:
   x. the isolated antibodies with increased Fc sialylation or fucosylation are subjected to antibody modification; and/or
   y. the isolated antibodies are subjected to depletion of IgG4 antibodies; and/or
   z. the isolated antibodies are subjected to depletion of IgG2 antibodies; and/or
   aa. the isolated antibodies are subjected to enrichment of antibodies with active effector function.

In some embodiments, the endogenous anti-tumor antibodies weakly induce tumor cell killing in vitro and/or are ineffective at or prevent tumor elimination in vivo. In some embodiments, the endogenous anti-tumor antibodies have defective Fc effector function or suppress IgG effector function.

In some embodiments, the extent of Fc sialylation is determined by Siglec-7 binding. In some embodiments, the extent of Fc sialylation is determined by Siglec-3 binding. In some embodiments, the extent of Fc sialylation is determined by Sambucus nigra agglutinin (SNA) binding. In some embodiments, the extent of Fc fucosylation is determined by FcγRIIa and FcγRIIIa interaction. In some embodiments, the extent of Fc fucosylation is determined by FcγRIIa interaction. In some embodiments, the extent of Fc fucosylation is determined by FcγRIIIa interaction. In some embodiments, the extent of FcγRIIIa interaction is determined by receptor binding. In some embodiments, the extent of Fc fucosylation is determined by the ratio of FcγRIIIa to FcγRIIa interaction. In some embodiments, the extent of FcγRIIa interaction is determined by receptor binding. In some embodiments, the extent of FcγRIIb interaction is determined by receptor binding. In some embodiments, the extent of FcγRIV interaction is determined by receptor binding.

In some embodiments, the tumor binding property of each antibody type and/or subtype is determined. In some embodiments, effector functional attributes are among cell killing, phagocytosis, complement response, cytokine release, and vaccination. In some embodiments, the bodily fluid is blood, plasma, serum, ascites or pleural effusion.

In some embodiments, the subjected to antibody modification comprises desialylating the antibodies, such as by using a neuraminidase. In some embodiments, the subjected to antibody modification comprises defucosylating the antibodies such as by using alpha-fucosidase. In some embodiments, enrichment of antibodies comprises enrichment or increasing of antibodies with active effector function. In some embodiments, enrichment of antibodies comprises increasing IgG1 and/or IgG3. In some embodiments, enrichment of antibodies comprises increasing the ratio of IgG1 and/or IgG3 to IgG2 and/or IgG4. In some embodiments, enrichment of antibodies comprises increasing the ratio of IgG1 to IgG2. In some embodiments, increasing the ratio comprises increasing the ratio of IgG1 to IgG4. In some embodiments, increasing the ratio comprises increasing the ratio of IgG3 to IgG2. In some embodiments, increasing the ratio comprises increasing the ratio of IgG3 to IgG4. In some embodiments, increasing the ratio comprises increasing the ratio of the combination of IgG1 and IgG3 to the combination of IgG2 and IgG4. In some embodiments, depletion of antibodies comprises depletion of IgG2 and/or IgG4. In some embodiments, depletion of antibodies with defective effector function comprises decreasing IgG2 and/or IgG4. In some embodiments, depletion of antibodies comprises decreasing the ratio of IgG2 and/or IgG4 to IgG 1 and/or IgG3. In some embodiments, depletion or antibodies comprises decreasing the ratio of IgG2 to IgG1. In some embodiments, depletion or antibodies comprises decreasing the ratio of IgG2 to IgG3. In some embodiments, depletion or antibodies comprises decreasing the ratio of IgG4 to IgG1. In some embodiments, depletion or antibodies comprises decreasing the ratio of IgG4 to IgG3. In some embodiments, depletion or antibodies comprises decreasing the ratio of the combination of IgG2 and IgG4 to the combination of IgG1 and IgG3. In some embodiments, the isolated antibodies are enriched for IgG1. In some embodiments, the isolated antibodies are enriched for IgG3. In some embodiments, enrichment of antibodies comprises increasing the IgG1 to IgG2 ratio. In some embodiments, the isolated antibodies are enriched for IgG1 over IgG2 or IgG4.

In one aspect, a method is provided for treating a subject having a tumor and endogenous anti-tumor antibodies comprising enhancing the anti-tumor efficacy of the endogenous anti-tumor antibodies from the subject as described in the foregoing aspect and administering the modified antibodies to the subject. In some embodiments, the modified anti-tumor antibodies are administered parenterally or intratumorally. In some embodiments, the treatment shrinks or eliminates the tumor.

In one aspect, a method is provided for treating a subject having a tumor and endogenous anti-tumor antibodies comprising enhancing the anti-tumor efficacy of the endogenous anti-tumor antibodies from the subject as described in the foregoing aspect, by administering to the subject one or more enzymes that modify the anti-tumor antibodies in vivo or in situ. In some embodiments, the treatment shrinks or eliminates the tumor. In some embodiments, the enzyme is a desialylase such as neuraminidase or a defucosylase such as alpha-fucosidase. In some embodiments, two or more enzymes are administered.

In one aspect, a method is provided for treating a subject having a tumor and endogenous anti-tumor antibodies, the method comprising the steps of:
a. obtaining a sample of the tumor and/or a bodily fluid from the subject;
b. isolating antibodies bound to the tumor and/or antibodies in the bodily fluid that bind to the tumor;
c. identifying and quantifying antibody types among the isolated antibodies;
d. identifying and quantifying IgG antibody subtypes among any IgG antibodies therein; and
e. identifying among each of the antibody types and/or subtypes therein one or more Fc properties comprising from among:
   1. extent of Fc sialylation, wherein increased Fc sialylation indicates defective IgG effector function or active suppression of IgG effector function;
   2. extent of Fc fucosylation, wherein increased Fc fucosylation indicates defective IgG effector function;
   3. extent of FcγRIIa interaction, wherein decreased FcγRIIa interaction indicates defective IgG effector function; and/or
   4. extent of FcγRIIIa interaction, wherein decreased FcγRIIIa interaction indicates defective IgG effector function; and/or
   5. extent of FcγRIIb interaction, wherein increased FcγRIIb interaction indicates defective IgG effector function; and/or
   6. extent of FcγRIV interaction, wherein decreased FcγRIV interaction indicates defective IgG effector function; and/or
   7. extent of IgG4, wherein high levels of or enrichment in IgG4 indicates a deficient anti-tumor antibody response; and/or
   8. extent of IgG2, wherein high levels of or enrichment in IgG2 indicates a deficient anti-tumor antibody response; and
f. optionally identifying and quantifying among the isolated antibodies any such antibodies that recognize specific tumor markers on the tumor with the properties above delineated for each marker;
wherein
   x. modifying the isolated antibodies with increased Fc sialylation or fucosylation by antibody modification; and/or
   y. modifying the isolated antibodies by depletion of IgG4 antibodies; and/or
   z. modifying the isolated antibodies by depletion of IgG2 antibodies; and/or
   aa. modifying the isolated antibodies by enrichment of antibodies with active effector function; and
administering to the subject any one or more of the modified antibodies.

In some embodiments, the endogenous anti-tumor antibodies weakly induce tumor cell killing in vitro and/or are ineffective at or prevent tumor elimination in vivo. In some embodiments, the endogenous anti-tumor antibodies have defective Fc effector function or suppress IgG effector function.

In some embodiments, the extent of Fc sialylation is determined by Siglec-7 binding. In some embodiments, the extent of Fc sialylation is determined by Siglec-3 binding. In some embodiments, the extent of Fc sialylation is determined by Sambucus nigra agglutinin (SNA) binding. In some embodiments, the extent of Fc fucosylation is determined by FcγRIIa and FcγRIIIa interaction. In some embodiments, the extent of Fc fucosylation is determined by FcγRIIa interaction. In some embodiments, the extent of Fc fucosylation is determined by FcγRIIIa interaction. In some embodiments, the extent of FcγRIIIa interaction is determined by receptor binding. In some embodiments, the extent of Fc fucosylation is determined by the ratio of FcγRIIIa to FcγRIIa interaction. In some embodiments, the extent of FcγRIIa interaction is determined by receptor binding. In some embodiments, the extent of FcγRIIb interaction is determined by receptor binding. In some embodiments, the extent of FcγRIV interaction is determined by receptor binding.

In some embodiments, the tumor binding property of each antibody type and/or subtype is determined. In some embodiments, effector functional attributes are among cell killing, phagocytosis, complement response, cytokine release, and vaccination. In some embodiments, the bodily fluid is blood, plasma, serum, ascites or pleural effusion.

In some embodiments, modifying the isolated antibodies comprises defucosylating the antibodies, such as alpha-fucosidase. In some embodiments, modifying the isolated antibodies comprises desialylating the antibodies, such as by using a neuraminidase. In some embodiments, modifying the isolated antibodies comprises enrichment of antibodies with active effector function. In some embodiments, enrichment of antibodies comprises increasing the IgG3 to IgG4 ratio. In some embodiments, isolated antibodies are enriched for IgG3. In some embodiments, enrichment of antibodies comprises increasing the IgG1 to IgG2 ratio. In some embodiments, isolated antibodies are enriched for IgG1 over IgG2 or IgG4.

In some embodiments, enrichment of antibodies comprises enrichment or increasing of antibodies with active effector function. In some embodiments, enrichment of antibodies comprises increasing IgG1 and/or IgG3. In some embodiments, enrichment of antibodies comprises increasing the ratio of IgG1 and/or IgG3 to IgG2 and/or IgG4. In some embodiments, enrichment of antibodies comprises increasing the ratio of IgG1 to IgG2. In some embodiments, increasing the ratio comprises increasing the ratio of IgG1 to IgG4. In some embodiments, increasing the ratio comprises increasing the ratio of IgG3 to IgG2. In some embodiments, increasing the ratio comprises increasing the ratio of IgG3 to IgG4. In some embodiments, increasing the ratio comprises increasing the ratio of the combination of IgG1 and IgG3 to the combination of IgG 2 and IgG4. In some embodiments, depletion of antibodies comprises depletion of IgG2 and/or IgG4. In some embodiments, depletion of antibodies with defective effector function comprises decreasing IgG2 and/or IgG4. In some embodiments, depletion of antibodies comprises decreasing the ratio of IgG 2 and/or IgG4 to IgG 1 and/or IgG3. In some embodiments, depletion or antibodies comprises decreasing the ratio of IgG2 to IgG1. In some embodiments, depletion or antibodies comprises decreasing the ratio of IgG2 to IgG3. In some embodiments, depletion or antibodies comprises decreasing the ratio of IgG4 to IgG1. In some embodiments, depletion or antibodies comprises decreasing the ratio of IgG4 to IgG3. In some embodiments, depletion or antibodies comprises decreasing the ratio of the combination of IgG2 and IgG4 to the combination of IgG1 and IgG3. In some embodiments, the isolated antibodies are enriched for IgG1. In some embodiments, the isolated antibodies are enriched for IgG3.

In some embodiments, the treatment shrinks or eliminates the tumor.

In one aspect, a method is provided for treating a subject having a tumor and endogenous anti-tumor antibodies, comprising administering an enzyme that desialylates antibodies, such as a neuraminidase, intratumorally or in proximity to the tumor. In some embodiments, the endogenous anti-tumor antibodies have increased Fc sialylation.

In one aspect, a method is provided for treating a subject having a tumor and endogenous anti-tumor antibodies, comprising administering an enzyme that defucosylates the antibodies, such as an alpha-fucosidase, intratumorally or in proximity to the tumor. In some embodiments, the endogenous anti-tumor antibodies have increased Fc fucosylation.

In one aspect, a method is provided for treating a subject having a tumor and endogenous anti-tumor antibodies, the method comprising the steps of:
a. obtaining a sample of the tumor and/or a bodily fluid from the subject;
b. isolating antibodies bound to the tumor and/or antibodies in the bodily fluid that bind to the tumor;
c. identifying and quantifying antibody types among the isolated antibodies;
d. identifying among each of the antibody types and/or subtypes therein one or more Fc properties comprising from among:
   1. extent of Fc sialylation, wherein increased Fc sialylation indicates defective IgG effector function or active suppression of IgG effector function;
   2. extent of Fc fucosylation, wherein increased Fc fucosylation indicates defective IgG effector; and/or
   3. extent of FcγRIIa interaction, wherein decreased FcγRIIa interaction indicates defective IgG effector function; and/or
   4. extent of FcγRIIIa interaction, wherein decreased FcγRIIIa interaction indicates defective IgG effector function; and/or
   5. extent of FcγRIIb interaction, wherein increased FcγRIIb interaction indicates defective IgG effector function; and/or
   6. extent of FcγRIV interaction, wherein decreased FcγRIV interaction indicates defective IgG effector function; and/or
and administering an enzyme, such as a desialylase such as neuraminidase or a defucosylase such as alpha-fucosidase, to modify endogenous anti-tumor antibodies in situ, intratumorally or proximal to the tumor.

In some embodiments of the prior two aspects, the endogenous anti-tumor antibodies weakly induce tumor cell killing in vitro and/or are ineffective at or prevent tumor elimination in vivo. In some embodiments, the endogenous anti-tumor antibodies have defective Fc effector function or suppress IgG effector function. In some embodiments, the enzyme is administered intratumorally or peritumorally.

In some embodiments, the extent of Fc sialylation is determined by Siglec-7 binding. In some embodiments, the extent of Fc sialylation is determined by Siglec-3 binding. In some embodiments, the extent of Fc sialylation is determined by Sambucus nigra agglutinin (SNA) binding. In some embodiments, the extent of Fc fucosylation is determined by the ratio of FcγRIIIa to FcγRIIa interaction. In some embodiments, the extent of FcγRIIa interaction is determined by receptor binding. In some embodiments, the extent of FcγRIIb interaction is determined by receptor binding. In some embodiments, the extent of FcγRIIIb interaction is determined by receptor binding. In some embodiments, the extent of FcγRIV interaction is determined by receptor binding. In some embodiments, the tumor binding property of each antibody type and/or subtype is determined. In some embodiments, effector functional attributes are among cell killing, phagocytosis, complement response, cytokine release, and vaccination. In some embodiments, the bodily fluid is blood, plasma, serum, ascites or pleural effusion.

In some embodiments, the treatment shrinks or eliminates the tumor. In some embodiments, the tumor is a solid tumor. In some embodiments, the tumor is high grade serous ovarian cancer.

### BRIEF DESCRIPTION OF THE DRAWINGS

FIG. 1 depicts tumor-associated antigens showing an IgG4-biased antibody response in ascites fluid. HER2, EGFR, and MMP14 are tumor-associated antigens. OC43 is a common cold coronavirus against which all samples had positive reactions. All patients shown were vaccinated and boosted against SARS-COV-2; therefore, SARS-COV-2 demonstrates a positive-control IgG4-biased response. Pan-IgG shows the signal from capturing IgG overall. N=5 samples from separate patients. Error bars indicate standard error of the mean.

FIG. 2 shows that antibodies against tumor-associated antigens have a selective loss of FcγRIIIa interaction. N=10 samples from separate patients (serum or ascites fluid). Circles indicate individual measurements, and the x indicates the mean across samples. Two samples that were negative for SARS-COV-2 antibodies were not plotted for that antigen.

FIG. 3 depicts MMP14 antibody responses showing higher amounts of Siglec-7 interaction in a subset of patients positive for anti-MMP14 antibodies. N=6 samples from separate patients (serum or ascites fluid). Circles indicate individual measurements, and the x indicates the mean across samples. IgG shows the pan-IgG response from capturing IgG overall.

FIG. 4 depicts the ratio of FcγRIIIa to FcγRIIa binding of IgG isolated by binding to OVCAR3 cells. This binding ratio is compared to IgG isolated by interaction with SARS-CoV-2 Spike, OC43 Spike, or EBV gp350. Average and standard deviation of measurements is also plotted alongside the individual measurements. Each point indicates one patient.

FIG. 5 is a scores plot for the principal components analysis of the OVCAR3-isolated IgG measurements. Each point indicates an individual patient.

FIG. 6 is a loadings plot for the principal components analysis of the OVCAR3-isolated IgG measurements. Each point indicates a type of IgG Fc feature that was quantified.

### DETAILED DESCRIPTION

Antibody (Ab) responses are widespread and abundant against many tumor types. Anti-tumor antibodies (ATAbs) display features of an active humoral immune response, including affinity enrichment through somatic hypermutation and high tumor selectivity. However, despite all the necessary components for eliminating cancer cells, ATAbs only weakly induce tumor cell killing in vitro and are ineffective at or prevent tumor elimination in vivo. This disclosure is directed to identifying defects in ATAbs and methods for correcting such defects, such that tumor elimination in vivo can be enhanced.

The present disclosure in some embodiments identifies, and in other embodiments corrects, defects in the Fc properties of ATAbs that underly an ineffective tumor immune response by an otherwise abundant ATAb population. Such Fc properties are an overlooked but, as will be described herein, important part of tumor immune evasion. Moreover, methods are described to correct Fc defects in a patient's ATAbs, comprising any one of: modifying Fc glycosylation, enriching or altering antibody populations to overcome Fc defects, or any combination thereof, among others. Such modifying, such as but not limited to modifying glycosylation and/or altering antibody subtype amounts or ratios, can be performed on antibodies ex vivo for subsequent infusion into the patient. In other embodiments, an enzyme capable of modifying antibody glycosylation to improve Fc effector function can be administered intratumorally or peritumorally, for therapeutic benefit.

As will be seen in the examples herein, a multiplexed approach was undertaken to profile antibody type, subclass, and biophysical interaction with Fc receptors across viral and common tumor antigens in high grade serous ovarian cancer (HGSOC), a non-limiting example of a tumor amenable to enhanced treatment by the methods disclosed herein. In parallel, ATAbs were isolated regardless of target antigen, through autologous tumor cell binding. The data revealed that ATAbs are substantially distinct in subclass composition and biophysical interactions: tumor responses were IgG4-biased, enriched in Siglec-7 interaction, and lacked FcγRIIIa interaction despite retained FcγRIIa binding. Comparing the tumor cell-fractionated samples to responses against the common tumor antigens EGFR, HER2, and MMP14, the results indicate that IgG4-enriched responses may be against host-shared antigens; tumor fractionization resulted in relatively more IgG3 and less IgG4. Anti-EGFR, Anti-HER2, and Anti-MMP14 IgG were absent from healthy subjects. These results indicate that despite active humoral immunity against HGSOC, specific mechanisms are blocking anti-tumor effector responses. The specific features implicate glycan changes in blocking antibody-dependent killing, in turn providing mechanisms of reactivating effector cell responses. The disclosure is directed to identifying and/or correcting such defects in the humoral anti-tumor immunity to activate effector responses to kill tumor cells. As will be described in further detail below, defective antibody effector functions resulting from altered Fc properties underly tumor immune evasion. These defects can be overcome by following the guidance herein.

Tumors. The methods disclosed herein are not limited to any particular type of tumor. In some embodiments, the tumor is a solid tumor. In some embodiments, the tumor is high grade serous ovarian cancer. In some embodiments, the tumor, suspected tumor, or resected tumor comprises a cancerous, pre-cancerous, or non-cancerous tumor. In some embodiments, the tumor comprises a sarcoma or a carcinoma, a fibrosarcoma, a myxosarcoma, a liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, an Ewing's tumor, a leiomyosarcoma, a rhabdomyosarcoma, a colon carcinoma, a pancreatic cancer or tumor, a breast cancer or tumor, an ovarian cancer or tumor, a prostate cancer or tumor, a squamous cell carcinoma, a basal cell carcinoma, an adenocarcinoma, a sweat gland carcinoma, a sebaceous gland carcinoma, a papillary carcinoma, a papillary adenocarcinomas, a cystadenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilm's tumor, a cervical cancer or tumor, a uterine cancer or tumor, a testicular cancer or tumor, a lung carcinoma, a small cell lung carcinoma, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a schwannoma, a meningioma, a melanoma, a neuroblastoma, or a retinoblastoma, esophageal cancer, pancreatic cancer, metastatic pancreatic cancer, metastatic adenocarcinoma of the pancreas, bladder cancer, stomach cancer, fibrotic cancer, glioma, malignant glioma, diffuse intrinsic pontine glioma, recurrent childhood brain neoplasm renal cell carcinoma, clear-cell metastatic renal cell carcinoma, kidney cancer, prostate cancer, metastatic castration resistant prostate cancer, stage IV prostate cancer, metastatic melanoma, melanoma, malignant melanoma, recurrent melanoma of the skin, melanoma brain metastases, stage IIIA skin melanoma; stage IIIB skin melanoma, stage IIIC skin melanoma; stage IV skin melanoma, malignant melanoma of head and neck, lung cancer, non-small cell lung cancer (NSCLC), squamous cell non-small cell lung cancer, breast cancer, recurrent metastatic breast cancer, hepatocellular carcinoma, Hodgkin's lymphoma, follicular lymphoma, non-Hodgkin's lymphoma, advanced B-cell NHL, HL including diffuse large B-cell lymphoma (DLBCL), multiple myeloma, chronic myeloid leukemia, adult acute myeloid leukemia in remission; adult acute myeloid leukemia with Inv(16)(p13.1q22); CBFB-MYH11; adult acute myeloid leukemia with t(16;16)(p13.1;q22); CBFB-MYH11; adult acute myeloid leukemia with t(8;21)(q22;q22); RUNX1-RUNX1T1; adult acute myeloid leukemia with t(9;11)(p22;q23); MLLT3-MLL; adult acute promyelocytic leukemia with t(15;17)(q22;q12); PML-RARA; alkylating agent-related acute myeloid leukemia, chronic lymphocytic leukemia, Richter's syndrome; Waldenstrom's macroglobulinemia, adult glioblastoma; adult gliosarcoma, recurrent glioblastoma, recurrent childhood rhabdomyosarcoma, recurrent Ewing sarcoma/peripheral primitive neuroectodermal tumor, recurrent neuroblastoma; recurrent osteosarcoma, colorectal cancer, MSI positive colorectal cancer; MSI negative colorectal cancer, nasopharyngeal nonkeratinizing carcinoma; recurrent nasopharyngeal undifferentiated carcinoma, cervical adenocarcinoma; cervical adenosquamous carcinoma; cervical squamous cell carcinoma; recurrent cervical carcinoma; stage IVA cervical cancer; stage IVB cervical cancer, anal canal squamous cell carcinoma; metastatic anal canal carcinoma; recurrent anal canal carcinoma, recurrent head and neck cancer; carcinoma, squamous cell of head and neck, head and neck squamous cell carcinoma (HNSCC), ovarian carcinoma, colon cancer, gastric cancer, advanced GI cancer, gastric adenocarcinoma; gastroesophageal junction adenocarcinoma, bone neoplasms, soft tissue sarcoma; bone sarcoma, thymic carcinoma, urothelial carcinoma, recurrent Merkel cell carcinoma; stage III Merkel cell carcinoma; stage IV Merkel cell carcinoma, myelodysplastic syndrome and recurrent mycosis fungoides and Sezary syndrome.

In one embodiment, high-grade serous ovarian carcinoma (HGSOC) is the most lethal gynecological malignancy, accounting for 70% of ovarian cancer mortality. Disease lethality is driven by rapid peritoneal tumor cell dissemination. The accumulation of fluid within the peritoneum, ascites, occurs in a third of patients on diagnosis and nearly all patients on therapeutic relapse. This fluid contains cancer cells that resist treatment and seed metastases. Thus, ascites is both an important route of tumor dissemination leading to mortality and sampling source for studying mechanisms of tumor malignancy. HGSOC cells are abundantly coated with autoantibodies with features of an active tumor immune response. Ascites is abundant in these Abs along with many immune cells capable of Ab-directed killing (e.g., NK cells, neutrophils). Despite all the necessary components, these Abs only weakly induce tumor cell killing in vitro and are ineffective at tumor elimination. In one embodiment, using ascites as a clinical sample source for the methods disclosed herein allows for the characterization of Fc properties of ineffective ATAbs, as well as means for modifying such ATAbs to enhance their effectiveness. Such methods, identifications of Fc properties and correcting them are applicable to any other tumor and such studies disclosed herein using HGSOC are merely exemplary and non-limiting.

Source of ATAbs. For characterizing and profiling ATAb Fc properties, antibody samples may be obtained from a bodily fluid such as blood or ascites, or antibodies may be isolated from tumor biopsy tissue.

The serum fraction prepared from whole blood can be used as the source of antibodies for the ensuing steps. Antibodies can be isolated from ascites by methods known in the art. In one exemplary method, serum or ascites is incubated with tumor cells, and unbound Abs washed away; tumor cells are then lysed to recover the bound Abs.

Isolation of antibodies bound to tumor. A tumor biopsy may provide a sample from which antibodies bound thereto may be isolated and characterized as described herein.

In another embodiment, ATAbs may be identified in a patient as associated with a prototypical tumor marker such as but not limited to EGFR, HER2, and/or NY-ESO-1. In one embodiment, where the patient's tumor comprises such one or more markers, methods herein that rely on a biopsy tumor sample may be carried out with a tumor cell line or non-tumor cell line expressing such marker, or non-cellular materials, e.g., beads, to which such marker is bound. Such cell lines and marker proteins are readily available. Thus, in embodiments that utilize tumor cells for antibody type, subtype and Fc property identification, methods for modifying antibodies, methods for testing such modified antibodies on tumor cells, and other quality systems or quality control processes needed on modified antibodies, may be carried out with any of these alternate forms of the patient's tumor cells that provide the same or similar purposes for the intended uses.

Isolation of ATAb Types and Subtypes. In one example, isolated ATAbs from bodily fluids or tumor cells will be incubated with magnetic beads conjugated with pan-IgG, pan-IgA, or pan-IgM Ab to immobilize each Ab type. The amount of antibody is quantified by receptor binding or other method, for example, using recombinant receptors. Such immobilized antibodies may then be further processed as described below, such as further characterization of antibody subtypes therein; characterization of Fc properties including but not limited to sialylation, fucosylation, FcγRIIa binding, FcγRIIIa binding, etc.

Such methods are merely exemplary of a variety of means whereby the isolation of ATAb types and subtypes may be accomplished. The skilled artisan will be aware of other methods, which are fully embraced by the present disclosure.

### Characterizing and Profiling Fc Properties of Isolated ATAbs

In some embodiments, the beads are subsequently deposited into separate wells and stained with a panel of detection reagents to quantify various Fc properties. Through the fluorescence signal between the different detection reagents and subjects, the pattern of Ab immune receptor interactions will be profiled.

In some embodiments, PE-conjugated anti-IgG1 Ab, anti-IgG1, anti-IgG2, anti-IgG3, anti-IgG4, FcγRI, FcγRIIa, FcγRIIIa, FcγRIIb, and/or FcγRIV are used to characterize the isolated IgG antibodies from the samples. PE labeled Fc-alpha/μR is used to characterize the isolated IgA and IgM antibodies.

In some embodiments, Ag-specific Ab-binding subsets (EGFR, HER2, Flu HA, OC43 Spike), or a pan-type averages (pan-IgG, pan-IgA, pan-IgM), will also be isolated in parallel directly from serum/ascites, using a standard systems serology approach. Such Ag-specific subsets may be used as a basis of comparison.

With regard to the distribution of a patient's ATAbs among the immunoglobulin types, the following descriptions with regard to ATAbs is exemplary and non-limiting.
IgA. IgA is highly effective in eliciting Fc effector functions. Enriching for IgA from ATAbs would be desirable for enhancing humoral immunity.
IgM. IgM is somewhat effective in eliciting Fc effector responses. However, it does not generally compete with other molecules in eliciting such responses.
IgG1. IgG1 is productive in eliciting Fc effector functions. Enriching for IgG1 over IgG2 or IgG4 is desirable for enhancing humoral immunity.
IgG2. IgG2 has reduced Fc effector functions and reducing or eliminating IgG2 from ATAbs is desirable in enhancing humoral immunity.
IgG3. IgG3 has heightened Fc effector functions. Enriching for IgG3 from ATAbs would be desirable for enhancing humoral immunity.
IgG4. IgG4 has reduced Fc effector functions and reducing or eliminating IgG4 from ATAbs is desirable in enhancing humoral immunity.
FcγRI. FcγRI is thought to be a high-affinity receptor for many different types of ATAbs.
FcγRIIa. FcγRIIa is effective in promoting the development of T cell immunity and phagocytotic Fc effector responses. Enriching for ATAbs that have this interaction, such as IgG1, would be desirable for enhancing humoral immunity.
FcγRIIIa. FcγRIIIa is highly effective in eliciting cell killing Fc effector functions. Enriching for ATAbs that have this interaction, such as IgG3 or afucosylated IgG, would be desirable for enhancing humoral immunity.
FcγRIIb. FcγRIIb is an inhibitory receptor that modulates the immune responses of several innate immune cell types. Reducing the interaction of ATAbs with this receptor would be desirable for enhancing tumor immunity.
FcγRIV. FcγRIV is effective in promoting phagocytic and cytokine responses to antibodies in certain tissues. Enriching for ATAbs that have this interaction would be desirable for enhancing tumor immunity.
Fc-alpha/μR. Fc-alpha/μR is highly effective in eliciting Fc effector functions by neutrophils. Enriching for ATAbs that have this interaction, such as by enriching the amount of IgA1 over IgA2, would be desirable for enhancing humoral immunity.

Determining ratio of IgG4 and/or IgG2 to IgG3 and/or IgG1. Quantification of the types and subtypes of immunoglobulins in a patient's ATAbs is used to provide ratios of various types and subtypes, for example, the ratio of IgG4 and/or IgG2 to IgG3 and/or IgG1. A high ratio indicates a defective effector function because IgG4 and IgG2 have reduced effector function. As described further below, a strategy to overcome reduced effector function because of a high IgG4 and/or IgG 2/IgG3 and/or IgG1 ratio is to deplete ATAbs of IgG4 and/or IgG2. Thus, in other embodiments, defective effector functions may be attributed to a ratio of a high IgG4/IgG3, IgG4/IgG1, IgG2/IgG1 and/or IgG2/IgG3. In other embodiments, defective effector functions may be attributed to a ratio of a high (IgG4+IgG2)/IgG1, (IgG4+IgG2)/IgG3, IgG4/(IgG1+IgG3), IgG2/(IgG1+IgG3), (IgG4+IgG2)/(IgG1+IgG3), or by determining any combination of such ratios.

Determining Subtype Ratios. Quantification of the types and subtypes of immunoglobulins in a patient's ATAbs is used to provide ratios of various types and subtypes, for example, the ratio of IgG4/IgG3, IgG4/IgG1, IgG2/IgG1, IgG2/IgG3, (IgG4+IgG2)/IgG1, (IgG4+IgG2)/IgG3, IgG4/(IgG1+IgG3), IgG2/(IgG1+IgG3), or (IgG4+IgG2)/(IgG1+IgG3). A high ratio indicates a defective effector function because IgG4 and IgG2 have reduced effector function. As described below, a strategy to overcome reduced effector function because of a high ratio is to deplete ATAbs of IgG2 and/or IgG4.

Detecting Altered Sialylation. In one embodiment, increased sialylation inhibits IgG Fc effector function. Identification of increased sialylation provides a rationale for treating a patient's ATAbs to decrease sialylation, as described below. Detection of sialylation may be performed by assessing binding of Siglec-7, a sialic acid-binding lectin. Various labeled or detectable Siglec-7 reagents are available for such purposes (e.g., a biotinylated Siglec-7). In one embodiment, beads to which ATAbs are bound are probed with a Siglec-7 reagent. As described below, a strategy to overcome reduced effector function due to high sialylation is to treat ATAbs with a sialylase. In other embodiments, extend of sialylation is measured by Siglec-3 binding or SNA binding, for which detectable reagents are available for assessing binding.

Detecting Altered Fucosylation. In one embodiment, decreased Fc fucosylation increases cell killing by antibody-dependent cellular cytotoxicity. Binding of antibodies to FcγRIIIa or FcγRIIa is a fucosylation-responsive activity. Detecting extent of fucosylation of ATAbs, compared to binding of FcγRIIIa, indicates defective Fc properties caused by increase fucosylation. As will be described below, in one embodiment, defucosylation of ATAbs increases Fc effector functions. See for example Li et al., “Chemoenzymatic Defucosylation of Therapeutic Antibodies for Enhanced Effector Functions Using Bacterial α-Fucosidases,” Methods Mol Biol 2018:1827:367-380.

Identifying Prototypical Antibodies in a Patient's ATAbs. As disclosed herein, identifying a patient's tumor as having a prototypical marker, such as a well-characterized tumor marker for which diagnostic and/or therapeutic ligands have been developed and may be readily available, provides a ready means for studying Fc effector defects in the ATAb population specific for the tumor and evaluating methods disclosed herein for altering effector functions.

### Analysis Methods and Alternate Methods

The aforementioned description and methods provide exemplary means for characterizing the properties of a patient's ATAbs to identify one or more reasons why the humoral antibody response in a patient is insufficient to trigger effector responses and induce tumor killing. While such determination of the rationale for a particular patient's defective immune response is informative, such determination is optional for the subsequent modification of the patient's ATAbs for enhancing effector functions. In some embodiments, an ATAb modification protocol may be carried out regardless of determining one or more of Fc characteristics, or such modification may be guided by a screening test for ATAb antibody types; or such modification may be guided by a screening test for ATAb antibody subtypes; or such modification may be guided by a screening test for only one ATAb antibody subtype for glycosylation; or such modification may be guided by a screening test for only one ATAb antibody subtype for sialylation; or such modification may be guided by a screening test for only one ATAb antibody subtype for fucosylation. Thus, guidance for the modification of a patient's ATAbs may be made based on no information about the Ig types, subtypes or Fc properties, or be made based on a single assessment, or on more than one assessment or a panel of assessments. Such assessments are non-limiting as to the predecessor to ATAb modification.

The foregoing and ensuing description of methods for assessing ATAb Fc properties may also be used for characterizing ATAbs after they are subjected to the modifications described below and elsewhere herein. Such methods for characterizing may provide means for qualifying whether the modification process has achieved the desired extent in vitro, ex vivo or in situ, based on future establishment of a relationship with the needed extent of Fc defect correction among individual and overall antibody types and subtypes, and clinical efficacy. Any such qualification, such as but not limited to percent of each antibody type or subtype modified, concentration of modified antibody, remaining extent of sialylation and/or fucosylation, alteration in receptor binding properties, individual antibody subtype content or ratios among any antibody type or subtype in the modified ATAbs, duration or enzyme treatment, among other means for process quality control or acceptability for administration, are fully embraced by the present disclosure. Such assessments are not intended to be limited to any particular methods and may be guided by regulatory requirements yet to be established.

### Modification of ATAb Fc Properties

As noted herein, Fc property defects of a patient's ATAbs may arise from alterations in the glycosylation pattern of the Fc region of one or more immunoglobulin (types or subtypes), and/or may derive from an altered ratio or different immunoglobulin types or subtypes among the patient's ATAbs (which in some embodiments have different Fc effector functions). Thus, depending on the method for improving ATAb effector functions guided by the disclosure herein, different methods may be employed. Non-limiting examples of such methods are described below; others may be readily gleaned from the teachings herein. Any such methods are embraced as modifications, such as but not limited to alteration in glycosylation, alteration in ratios, alteration in amounts, etc.

### Altering Ratios of Immunoglobulin Types

Altering Ratios of Immunoglobulin Subtypes. As noted herein, IgG4 has reduced effector function compared with IgG3. In one embodiment, a high IgG4/IgG3 ratio indicates a defective Fc effector function by reason of high IgG4. In one embodiment, a patient having a high IgG4/IgG3 ratio may have ATAbs de-enriched for IgG4 by a method such as but not limited to using anti-IgG4 selective antibodies.

Also as noted herein, IgG2 has reduced effector function compared with IgG1 or IgG3. In one embodiment, a high IgG2/IgG1 ratio indicates a defective Fc effector function by reason of high IgG2. In one embodiment, a patient having a high IgG2/IgG1 ratio may have ATAbs de-enriched for IgG2 by a method such as but not limited to using anti-IgG2 selective antibodies.

Moreover, enriching for IgG1 over IgG2 or IgG4 is desirable. Enriching for IgG3 over IgG2 or IgG4 is desirable.

Also as noted herein, desirable enrichment of antibodies to enhance effector function comprises enriching for IgG1 and/or IgG3, and/or de-enriching for IgG2 and/or IgG4. Thus, in one embodiment, the method comprises enriching IgG1. In one embodiment, the method comprises enriching in IgG3. In one embodiment, the method comprises enriching in IgG1 and IgG3. In one embodiment, the method comprises de-enriching for IgG2. In one embodiment, the method comprises de-enriching for IgG4. In one embodiment, the method comprises de-enriching for IgG2 and IgG4. In some embodiments, enrichment of antibodies comprises increasing IgG1 and/or IgG3. In some embodiments, enrichment of antibodies comprises increasing the ratio of IgG1 and/or IgG3 to IgG2 and/or IgG4. In some embodiments, enrichment of antibodies comprises increasing the ratio of IgG1 to IgG2. In some embodiments, increasing the ratio comprises increasing the ratio of IgG1 to IgG4. In some embodiments, increasing the ratio comprises increasing the ratio of IgG3 to IgG2. In some embodiments, increasing the ratio comprises increasing the ratio of IgG3 to IgG4. In some embodiments, increasing the ratio comprises increasing the ratio of the combination of IgG1 and IgG3 to the combination of IgG2 and IgG4. In some embodiments, depletion of antibodies comprises depletion of IgG2 and/or IgG4. In some embodiments, depletion of antibodies with defective effector function comprises decreasing IgG2 and/or IgG4. In some embodiments, depletion of antibodies comprises decreasing the ratio of IgG2 and/or IgG4 to IgG1 and/or IgG3. In some embodiments, depletion or antibodies comprises decreasing the ratio of IgG2 to IgG1. In some embodiments, depletion or antibodies comprises decreasing the ratio of IgG2 to IgG3. In some embodiments, depletion or antibodies comprises decreasing the ratio of IgG4 to IgG1. In some embodiments, depletion or antibodies comprises decreasing the ratio of IgG4 to IgG3. In some embodiments, depletion or antibodies comprises decreasing the ratio of the combination of IgG2 and IgG4 to the combination of IgG1 and IgG3.

Altering Sialylation of Immunoglobulin Types and Subtypes. In one embodiment, IgG from isolated ATAbs is desialylated using a neuraminidase. By way of non-limiting example, a bacterial enzyme may be used. In some embodiments, the neuraminidase may be bound to beads such as magnetic beads, to facilitate ex vivo treatment of antibodies. In some embodiments, a human neuraminidase such as a recombinant human enzyme may be used for the in-situ treatment described elsewhere herein.

Altering Fucosylation of Immunoglobulin Types and Subtypes. In one embodiment, antibodies are desialylated using a bacterial alpha-fucosidase (e.g., alpha-(1,6) fucosidase). In some embodiments, the fucosidase may be bound to beads such as magnetic beads to facilitate ex vivo treatment of antibodies. In one embodiment, defucosylation of antibodies is achieved in situ, such as using a recombinant enzyme as described for neuraminidase, In some embodiments, a defucosylating enzyme such as but not limited to alpha-fucosidase may be administered. Administration to the site (e.g., in situ) may be guided by ultrasound or other means. Administration may be by bolus or infusion. Such in situ modification of defective Fc effector function may be carried out with, in one embodiment, alpha-fucosidase provided as a lyophilized, sterile powder with appropriate excipients in vials, for reconstitution with sterile water and infusion to the patient. Other forms and types of defucosylating enzymes and other in-situ treatments for increased antibody Fc effector function are fully embraced herein.

Non-limiting examples of enzymes useful for the purposes herein include α1-2,4,6 fucosidase O (New England Biolabs); and a fucosidase described in Tsai et al., An Effective Bacterial Fucosidase for Glycoprotein Remodeling, ACS Chem. Biol. 2017, 12, 1, 63-72.

### Administration of Modified ATAbs to the Patient

After following one or more methods disclosed herein for enhancing Fc properties of ATAbs, the ATAbs may be infused into the patient and/or to the site of the tumor. Routes of parenteral administration include but are not limited to parenterally, subcutaneous, intramuscular, intravenous, intraarticular, intrabronchial, intraabdominal, intracapsular, intrachondral, intracavitary, intracelial, intracerebellar, intraventricular, intrathecal, intracolonic, intracervical, intragastric, intrahepatic, intramyocardial, intraosseous, intrapelvic, intrapericardial, intraperitoneal, intrapleural, intraprostatic, intrapulmonary, intrarectal, intrarenal, intraretinal, intrabursal, intrapleural, intrauterine, intravesical, intralesional, bolus, vaginal, rectal, buccal, sublingual, intranasal and transdermal.

ATAbs after modification as described herein are formulated in a suitable vehicle, buffer, excipient, or combination thereof, suitable for human administration. In some embodiments, modified ATABs are concentrated before formulation and/or administration. As noted above, the modified ATABs may be qualified by analysis during processing and in the formulation to be administered.

### Administration of Desialylating Enzymes to the Patient

In one embodiment, defective Fc effector functions of ATAbs in vivo may be enhanced by administration of enzymes that desialylate IgG to the tumor, such as intratumorally or in the vicinity of the tumor (e.g., in situ). In some embodiments, a recombinant human neuraminidase may be administered. Administration to the site may be guided by ultrasound or other means. Administration may be by bolus or infusion. Such in situ modification of defective Fc effector function may be carried out with, in one embodiment, recombinant human neuraminidase provided as a lyophilized, sterile powder with appropriate excipients in vials, for reconstitution with sterile water and infusion to the patient. Other forms and types of desialylating enzymes and other in-situ treatments for increased antibody Fc effector function are fully embraced herein.

In one embodiment, defective Fc effector functions of ATAbs in vivo may be enhanced by administration of enzymes that defucosylate IgG to the tumor, such as intratumorally or in the vicinity of the tumor (e.g., in situ). In some embodiments, alpha-fucosidase may be administered. In some embodiments, recombinant enzymes are administered. Administration to the site may be guided by ultrasound or other means. Administration may be by bolus or infusion. Such in situ modification of defective Fc effector function may be carried out with, in one embodiment, alpha-fucosidase is provided as a lyophilized, sterile powder with appropriate excipients in vials, for reconstitution with sterile water and infusion to the patient. Other forms and types of defucosylating enzymes and other in-situ treatments for increased antibody Fc effector function are fully embraced herein.

### Assessment of Tumor Cell Killing

Tumor cell killing may be assessed by any number of means known to one of skill in the art, such as but not limited to an antibody-dependent cellular cytotoxicity (ADCC) assay, such as the methods described in Yeap et al., 2016, CD16 is indispensable for antibody-dependent cellular cytotoxicity by human monocytes, Scientific Reports volume 6, Article number: 34310 (2016 ), incorporated by reference herein.

### Subjects

In some embodiments, the methods disclosed herein are used for humans. In other embodiments, analogous methods may be used for assessing Fc effector function and treatment of non-human animals, such as for domesticated pets, farm or zoo animals. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys. The term “higher vertebrates” is used herein and includes avians (birds) and mammals. The methods and compositions described herein can be used to treat any suitable mammal, including primates, such as monkeys and humans, horses, cows, cats, dogs, rabbits, sheep, goats, pigs, and rodents such as rats and mice. In one embodiment, the mammal to be treated is human. The human can be any human of any age. In an embodiment, the human is an adult. In another embodiment, the human is a child. The human can be male, female, pregnant, middle-aged, adolescent, or elderly. According to any of the methods of the present invention and in one embodiment, the subject is human. In another embodiment, the subject is a non-human primate. In another embodiment, the subject is murine, which in one embodiment is a mouse, and, in another embodiment is a rat. In another embodiment, the subject is canine, feline, bovine, equine, laprine, or porcine. In another embodiment, the subject is mammalian.

### EXAMPLES

### Example 1. Patient Sample Dissociation and Tumor Cell Isolation

Tumor samples were washed twice with complete growth media (RPMI 1640 medium supplemented with 10% fetal bovine serum and 1% penicillin/streptomycin) and centrifuged at 500 g at room temperature, then resuspended in growth media. A 5× solution of collagenase I and dispase II (Thermo Scientific) was added to the resuspended sample for a final concentration of 2 mg/mL. Several flakes of DNase I (Sigma-Aldrich) were added to the cell suspension and incubated at 37 °C on a shaker for 1 hour. If large aggregates were still present, the cell suspension was re-digested for another hour. Cells were harvested by centrifugation at 500 g for 5 minutes.

An EasySep CD45 Depletion Kit (STEMCELL Technologies) was used to isolate the tumor cells. Recommended Medium was generated using PBS containing 2% FBS and 1 mM ethylenediaminetetraacetic acid (EDTA) (Fisher Scientific). The cells were resuspended at a volume of 1 million cells per 10 μL and transferred to a round bottom tube. 50 μL/mL of Depletion Cocktail from the kit was added, mixed, and incubated for 5 minutes at room temperature (RT). 75 μL/mL of vortexed RapidSpheres from the kit were added afterwards, mixed, and incubated for 3 minutes at RT. Recommended Medium was then added until a final volume of 2.5 mL was reached, mixed, and the tube was placed onto an EasySep magnet from the kit for 5 minutes. Then, in one motion, the magnet was inverted over secondary round bottom tube. The magnet was kept inverted for 2-3 seconds without wiping or tapping additional drops into the secondary tube. The secondary tube was then placed into the magnet for 5 minutes. Afterwards, the magnet was again inverted for 2-3 seconds to transfer the contents into a conical tube. Cells were then counted and transferred into 1.5 mL Eppendorf tubes.

### Example 2. Cell-Based Antibody Pulldown and ATAb Isolation

The cells were spun down at 500 g for 5 minutes at 4 °C, and then resuspended in 100 μL per 300,000 cells of matching patient supernatant and incubated for 1 hour at 4 °C. Afterwards, cells were centrifuged at 500 g for 5 minutes at 4 °C. The supernatant was then removed.

Cells were then washed with 1 mL of cold PBS to remove any leftover supernatant and centrifuged at 500 g for 5 minutes at 4 °C. This wash step was repeated three times. Afterwards, the cells were incubated with 80 μL of NP 40 lysis buffer per 500,000 cells, with a minimum volume of 40 μL, for 5 minutes at RT. They were then centrifuged at 12,000 rpm for 5 minutes at 4 °C. The supernatant was collected and transferred to a new tube as lysates. The lysates were then stored at −80 °C or used immediately.

### Example 3. Systems Serology Analysis

A half-area 96-well plate was washed with 1× tris-buffered saline containing 0.1% Tween (TBST) prior to use. The capture bead sets for the assay were generated by conjugating pan-IgG (SouthernBiotech #9052-08), COVID-19 (Sino Biological #40589-V08H26), OC43 (Sino Biological #40607-V08B-100), MMP14 (R&D Systems #918-MPN-010), EGFR (Sino Biological #10001-H08H-100), and HER2 (Sino Biological #10004-H08H-50) antibodies to magnetic beads1 (Bio-Rad). For optimal results, the plate contained at least 50 beads/well of each type.

Prior to use, each bead stock was vortexed thoroughly. Appropriate volumes of each bead stock were transferred and combined into a single Eppendorf tube. 50 μL/well of assay buffer (1% BSA in PBS) was then added to the single tube and mixed thoroughly by vortexing. 50 μL/well of the bead-assay buffer solution was then added to the first row of the plate and washed for a second time. 15 μL of patient sample lysates and 15 μL of assay buffer (1:1 ratio) were then added to the each well of the first row of the washed plate. Patient samples and beads were incubated together overnight at 130 rpm at 4 °C in the dark. For assays conducted using patient serum or supernatant, samples were added following three 100-fold serial dilutions in assay buffer.

Following the overnight incubation period, the combined mixture was split evenly across the plate rows by adding 50 μL of assay buffer to each well of the first row and then using a pipette to transfer the contents in equal amounts and then washed. For example, for an assay with 7 detections, the first row would be split evenly among 6 additional wells vertically.

Detection-assay buffer solutions were then added to each corresponding well at a volume of 12.5 μL/well. The total volume of detection-assay buffer used was 12.5 μL/well. Biotinylated IgG1 (SouthernBiotech) was diluted 1:1000. Phycoerythrin (PE)-conjugated IgG2, IgG3, IgG4 (SouthernBiotech) were diluted 1:200. FcγRIIa, and FcγRIIIa (R&D Systems) antibodies were diluted 1:200. Biotinylated Siglec-7 (Acro Biosystems) was diluted 1:126. The FcγRIIa, FcγRIIIa, and Siglec-7 detection reagents were separately incubated with streptavidin-phycoerythrin (SAPE) at a 4:1 molar ratio for 15 minutes at RT in the dark prior to addition to the sample plate to tetramerize.

The plate was then incubated at 130 rpm for 1 hour at RT in the dark. Afterwards, the plate was washed again in a plate washer. For sample wells receiving biotinylated IgG1 detection, an additional incubation period with SAPE-assay buffer solution at 130 rpm for 15 minutes at RT was included. The SAPE-assay buffer solution was generated by diluting SAPE 100-fold in 12.5 μL/well of assay buffer. The plate was washed a final time in the plate washer. 40 μL/well of assay buffer was added to the plate prior to analysis on a Luminex MagPIX.

### Results

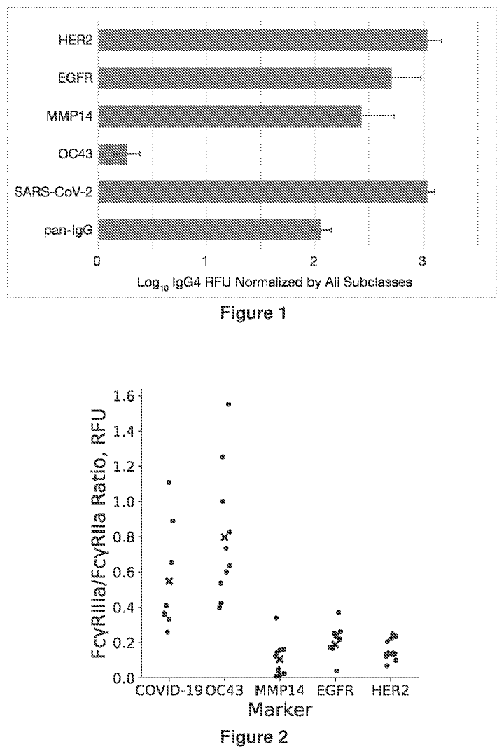

Tumor-associated antigens show an IgG4-biased antibody response in ascites fluid (FIG. 1). HER2, EGFR, and MMP14 are tumor-associated antigens. OC43 is a common cold coronavirus against which all samples had positive reactions. All patients were vaccinated and boosted against SARS-COV-2; therefore, SARS-COV-2 demonstrates a positive-control IgG4-biased response (see Irrgang et al., Class switch toward noninflammatory, spike-specific IgG4 antibodies after repeated SARS-COV-2 mRNA vaccination, Sci Immunol. 2023 January 27; 8(79):eade2798). Pan-IgG shows the signal from capturing IgG overall. N=5 samples from separate patients. Error bars indicate standard error of the mean.

FIG. 2 shows that antibodies against tumor-associated antigens have a selective loss of FcγRIIIa interaction. N=10 samples from separate patients (serum or ascites fluid). Circles indicate individual measurements, and the x indicates the mean across samples. Two samples that were negative for SARS-COV-2 antibodies were not plotted for that antigen.

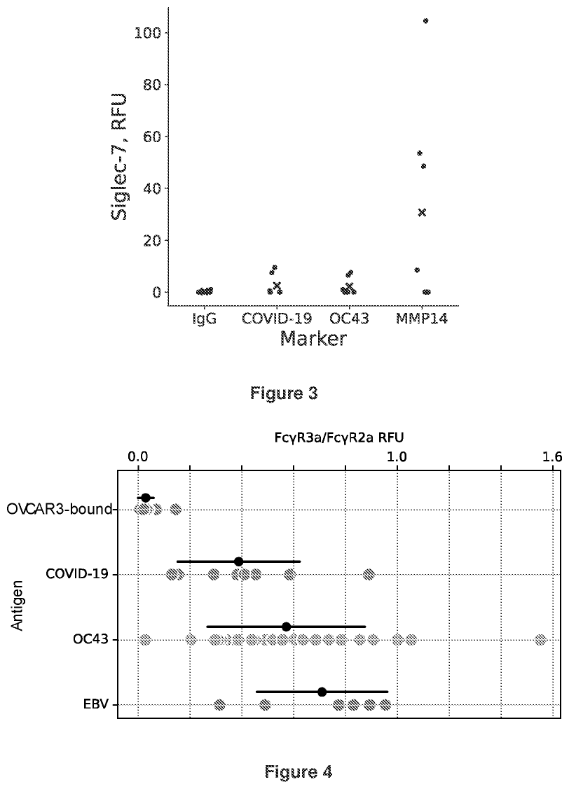

MMP14 antibody responses show higher amounts of Siglec-7 interaction in a subset of patients (FIG. 3). N=6 samples from separate patients (serum or ascites fluid). Circles indicate individual measurements, and the x indicates the mean across samples. IgG shows the pan-IgG response from capturing IgG overall.

### Example 4. Treating a Patient With High Grade Serous Ovarian Cancer (HGSOC) With ATAbs Desialylated ex Vivo

ATAbs are obtained from a patient with HGSOC, and defective Fc effector function are identified therein due to increased sialylation of IgG. ATAbs isolated from a tumor biopsy are treated with neuraminidase bound to magnetic beads, then isolated. Antibodies administered to the patient have enhanced Fc effector function.

Additional ATAbs may be collected from serum, similarly modified and infused into the patient.

### Example 5. Treating a Patient With High Grade Serous Ovarian Cancer (HGSOC) With ATAbs Enriched in IgG3

ATAbs are obtained from a patient with HGSOC, and defective Fc effector function are identified therein because of increased IgG4 levels. ATAbs isolated from a tumor biopsy are depleted of IgG4 using beads coated with anti-IgG4 antibody. Such antibodies administered to the patient have enhanced Fc effector function.

Additional ATAbs may be collected from serum, similarly enriched and infused into the patient.

### Example 6. Treating a Patient with High Grade Serous Ovarian Cancer (HGSOC) by Intratumoral Infusion of Neuraminidase

A patient with HGSOC is treated with recombinant human neuraminidase to increase Fc effector function in the tumor. A catheter is guided to the tumor site and a solution of neuraminidase is infused adjacent to the tumor. ATAbs in situ show increased Fc effector function.

### Example 7. Characterization of IgG That Interacts With Ovarian Cancer Cell Line OVCAR3

### Methods.

Cell-Based Antibody Pulldown and ATAb Isolation. OVCAR3 cells (NIH: OVCAR-3 [OVCAR3] (ATCC HTB-161)) were grown in complete growth media (RPMI 1640 supplemented with 20% fetal bovine serum, 1% penicillin streptomycin, and 1 mM sodium pyruvate). Patient ascites and serum were prepared by centrifugation at 12,000 rpm for 10 minutes at 4 °C, followed by a 1:1 dilution with assay buffer (1% bovine serum albumin in phosphate-buffered saline (PBS)). The cells were harvested and resuspended using the diluted patient ascites or serum at a concentration of 12.5 μL/ 125,000 cells, then added to a 96-well round-bottom non-tissue culture treated plate to be incubated for 60 minutes at 4 °C, gently shaking.

After the incubation, 100 μL of PBS was added to each well of the plate, followed by a centrifugation of the plate at 500 g for 5 minutes at 4 °C. The supernatant in each well was removed. The cell pellets were washed by resuspending in PBS. The centrifugation and PBS wash steps were repeated for a total of 7 times. Following the final wash, each cell pellet was resuspended in 50μL of an NP40 lysis buffer mixture with phosphatase and protease inhibitors and incubated on ice for 5-10 minutes, then spun down at 12,000 rpm for 10 minutes at 4 °C. The supernatants were collected and stored as lysates. These lysates containing the isolated ATAbs were further diluted with 50 μL of assay buffer prior to long-term storage at −80 °C or immediate use.

Systems Serology Analysis of Lysates. A 384-well plate was washed with 1× tris-buffered saline containing 0.1% Tween (TBS-T) prior to use. The capture bead sets for the assay were generated by conjugating pan-IgG (Southern Biotech #2043-01), pan-IgA (Bethyl Labs #A80-102), pan-IgM (Bethyl Labs #A80-100), COVID-19 (Sino Biological #40589-V08H26), OC43 (Sino Biological #40607-V08B-100), MMP14 (R&D Systems #918-MPN-010), EGFR (Sino Biological #10001-H08H-100), HER2 (Sino Biological #10004-H08H-50), Epstein-Barr virus (EBV) (Acro Biosystems #GP0-E52H6), p53 (R&D #SP-452-020), and cytomegalovirus (CMV) (Aviva Systems Bio #OPPA00462) antibodies/proteins to magnetic beads (Bio-Rad). For optimal results, the plate contained at least 50 beads/well of each type.

Prior to use, each bead stock was vortexed thoroughly. Appropriate volumes of each bead stock were transferred and combined into a single Eppendorf tube. 50 μL/well of assay buffer was then added to the single tube and mixed thoroughly by vortexing. 50 μL/well of the bead-assay buffer solution was then added to each well of the plate and washed for a second time. 10 μL of each patient sample lysate was then added to the each well of the washed plate. Patient samples and beads were incubated together overnight at 130 rpm at 4 °C in the dark. Following the overnight incubation period, the plate was washed.

Detection-assay buffer solutions were then added to each corresponding well at a volume of 6.25 μL/well. Biotinylated IgG1 (Southern Biotech #9052-08) was diluted 1:1000. Phycoerythrin (PE)-conjugated IgG2, IgG3, IgG4 (Southern Biotech #9070-09, #9210-09, #9200-09) were diluted 1:200. FcγRIIa and FcγRIIIa (R&D Systems #AVI1330-050, #AVI8894-050) proteins were diluted 1:200. Biotinylated Siglec-7 (Acro Biosystems #SG7-H82E7) was diluted 1:126. The FcγRIIa and FcγRIIIa and Siglec-7 detection reagents were separately incubated with streptavidin-phycoerythrin (SAPE) at a 4:1 molar ratio for 15 minutes at RT in the dark prior to addition to the sample plate to tetramerize.

The plate was then incubated at 130 rpm for 1 hour at RT in the dark. Afterwards, the plate was washed again. For sample wells receiving biotinylated IgG1 detection, an additional incubation period with SAPE-assay buffer solution at 130 rpm for 15 minutes at RT was included. The SAPE-assay buffer solution was generated by diluting SAPE 100-fold in 6.25 μL/well of assay buffer. The plate was washed a final time in the plate washer. 60 μL/well of assay buffer was added to the plate prior to analysis on a Luminex xMAP INTELLIFLEX System.

Antigen-Specific Antibody Analysis of Patient Samples. A 384-well plate was prepared as described earlier. Instead of using patient sample lysate, patient serum or ascites was diluted following three 100-fold serial dilutions in assay buffer and 5 μL of each dilution was added directly to the plate, in addition to 5 μL of assay buffer at a 1:1 ratio. Following the overnight incubation, detection-assay buffer solutions were added as described earlier and analyzed on the Luminex xMAP INTELLIFLEX System.

Results. In addition to characterizing IgG against specific antigen targets described above, an ovarian cancer cell line, OVCAR3, was used to isolate IgG that interacts with these cells. The OVCAR3-isolated fraction of IgG was then compared to IgG selective for three viral antigens. Anti-OVCAR3 IgG was observed to have a much lesser amount of FcγRIIIa interaction when normalized to FcγRIIa to correct for antibody amount (FIG. 4).

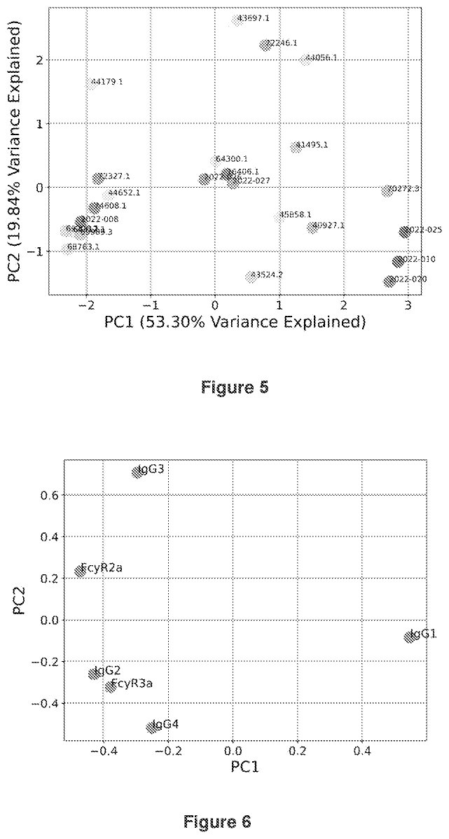

Comparing the full set of measurements by principal components analysis (PCA), there were not striking differences between the measurements collected at UCLA (labeled starting with 2022-) versus those at University of Wisconsin (FIG. 5). The variation among patients was by a first component that separated all measurements from those of IgG1, reflecting that a large amount of variation occurred in IgG1 amount without affecting the other measurements (FIG. 6). The second component then reflected that increasing IgG3 led to an increase in FcγRIIa interaction, but without changes in FcγRIIIa interaction.

Numerous modifications and variations in the disclosed methods as set forth in the above illustrative examples are expected to occur to those skilled in the art and are fully embraced by the disclosure herein.

What is claimed is:

1. A method for identifying an effective therapeutic regimen for a subject having a tumor and endogenous anti-tumor antibodies, the method comprising the steps of:
a. obtaining a sample of the tumor and/or a bodily fluid from the subject;
b. isolating antibodies bound to the tumor and/or antibodies in the bodily fluid that bind to the tumor;
c. identifying and quantifying antibody types among the isolated antibodies;
d. identifying and quantifying IgG antibody subtypes among any IgG antibodies therein; and
e. identifying among each of the antibody types and/or subtypes therein one or more Fc properties comprising from among:
1. extent of Fc sialylation, wherein increased Fc sialylation indicates defective IgG effector function or active suppression of IgG effector function; and/or
2. extent of Fc fucosylation, wherein increased Fc fucosylation indicates defective IgG effector; and/or
3. extent of FcγRIIa interaction, wherein decreased FcγRIIa interaction indicates defective IgG effector function; and/or
4. extent of FcγRIIIa interaction, wherein decreased FcγRIIIa interaction indicates defective IgG effector function; and/or
5. extent of FcγRIIb interaction, wherein increased FcγRIIb interaction indicates defective IgG effector function; and/or
6. extent of FcγRIV interaction, wherein decreased FcγRIV interaction indicates defective IgG effector function; and/or
7. extent of IgG4, wherein high levels of or enrichment in IgG4 indicates a deficient anti-tumor antibody response; and/or
8. extent of IgG2, wherein high levels of or enrichment in IgG2 indicates a deficient anti-tumor antibody response; and
wherein:
x. a defective antibody Fc effector function indicates a therapeutic regimen comprising administering endogenous anti-tumor antibodies after antibody modification thereof; and/or
y. a defective antibody Fc effector function indicates a therapeutic regimen comprising administering endogenous anti-tumor antibodies after depletion of antibodies with defective effector function; and/or
z. a defective antibody Fc effector function indicates a therapeutic regimen comprising administering endogenous anti-tumor antibodies after enrichment of antibodies with active effector function; and/or
aa. a defective antibody Fc effector function indicates a therapeutic regimen comprising administering an enzyme, such as a desialylase such as a neuraminidase, or a defucosylase, such as alpha-fucosidase, to modify endogenous anti-tumor antibodies in situ, localized to the tumor; and/or
bb. a deficient anti-tumor antibody response indicates a therapeutic regimen comprising depletion of IgG4 and/or enrichment in IgG3; and/or
cc. a deficient anti-tumor antibody response indicates a therapeutic regimen comprising depletion of IgG2 and/or IgG4 and/or enrichment in IgG1 and/over IgG3 over IgG2 and/or IgG4.

2. The method of claim 1, wherein the endogenous anti-tumor antibodies weakly induce tumor cell killing in vitro and/or are ineffective at or prevent tumor elimination in vivo.

3. The method of any one of claims 1-2, wherein the endogenous anti-tumor antibodies have defective Fc effector function or suppress IgG effector function.

4. The method of claim 1 wherein the extent of Fc sialylation is determined by Siglec-7, Siglec-3 or Sambucus nigra agglutinin (SNA) binding.

5. The method of claim 1 wherein the extent of Fc fucosylation is determined by FcγRIIa and FcγRIIIa interaction.

6. The method of claim 1 wherein the extent of FcγRIIa, FcγRIIb, and/or FcγRIIIa interaction is determined by receptor binding.

7. The method of claim 1 wherein the extent of FcγRIIIa interaction is determined by receptor binding. The method of claim 1 wherein the content of IgG4 is determined and/or a ratio of IgG4 to IgG3, to IgG1, and/or to the combination of IgG3 and IgG1 is determined, and/or the content of IgG2 is determined and/or a ratio of IgG2 to IgG1, to IgG3, and/or to the combination of IgG1 and IgG3 is determined.

9. The method of claim 1 wherein:
a. increased Fc sialylation indicates defective IgG effector function or active suppression of IgG effector function; and/or
b. increased Fc fucosylation indicates defective IgG effector function; and/or
c. decreased FcγRIIIa interaction indicates defective IgG effector function; and/or
d. decreased FcγRIIa interaction indicates defective IgG effector function; and/or
e. increased FcγRIIb interaction indicates defective IgG effector function; and/or
f. decreased FcγRIV interaction indicates defective IgG effector function; and/or
g. enriched IgG4 indicates defective anti-tumor antibody effector function;
and/or
h. high IgG4 content or ratio indicates a deficient anti-tumor antibody response; and/or
i. high IgG2 content or ratio indicates a deficient anti-tumor antibody response.

10. The method of claim 1 wherein the tumor binding property of each antibody type and/or subtype is determined.

11. The method of claim 1 further comprising identifying and/or quantifying among the isolated antibodies any such antibodies that recognize specific tumor markers on the tumor with the properties above delineated for each marker.

12. The method of claim 1 wherein the effector function attributes are determined before, during and/or after tumor therapy.

13. The method of claim 11 wherein tumor therapy comprises surgery, radiation, immunotherapy, chemotherapy, or any combination thereof.

14. The method of claim 1 wherein effector functional attributes are among cell killing, phagocytosis, complement response, cytokine release, and response to vaccination.

15. The method of claim 1 wherein the bodily fluid is blood, plasma, serum, ascites or pleural effusion.

16. The method of claim 1 wherein antibody modification comprises correcting defective antibody glycosylation.

17. The method of claim 15 wherein the correcting defective antibody glycosylation comprises desialylating the antibodies, such as by using a neuraminidase.

18. The method of claim 15 wherein the correcting defective antibody glycosylation comprises defucosylating the antibodies, such as by using alpha-fucosidase.

19. The method of claim 1 wherein the correcting defective Fc effector function comprises depleting IgG4 and/or depleting IgG2.

20. A method for treating a subject having a tumor and endogenous anti-tumor antibodies comprising identifying the effective therapeutic regimen as described in any one of claims 1-19, comprising modifying the endogenous anti-tumor antibodies to enhance anti-tumor activity, and administering the modified antibodies to the subject.

21. The method of claim 20 wherein the modified anti-tumor antibodies are administered parenterally or intratumorally.

22. A method for enhancing the anti-tumor efficacy of endogenous anti-tumor antibodies from a subject having a tumor and endogenous anti-tumor antibodies, the method comprising the steps of:
a. obtaining a sample of the tumor and/or a bodily fluid from the subject;
b. isolating antibodies bound to the tumor and/or antibodies in the bodily fluid that bind to the tumor;
c. identifying and quantifying antibody types among the isolated antibodies;
d. identifying and quantifying IgG antibody subtypes among any IgG antibodies therein; and
e. identifying among each of the antibody types and/or subtypes therein one or more Fc properties comprising from among:
1. extent of Fc sialylation, wherein increased Fc sialylation indicates defective IgG effector function or active suppression of IgG effector function; and/or
2. extent of Fc fucosylation, wherein increased Fc fucosylation indicates defective IgG effector; and/or
3. extent of FcγRIIa interaction, wherein decreased FcγRIIa interaction indicates defective IgG effector function; and/or
4. extent of FcγRIIIa interaction, wherein decreased FcγRIIIa interaction indicates defective IgG effector function; and/or
5. extent of FcγRIIb interaction, wherein increased FcγRIIb interaction indicates defective IgG effector function; and/or
6. extent of FcγRIV interaction, wherein decreased FcγRIV interaction indicates defective IgG effector function; and/or
7. extent of IgG4, wherein high levels of or enrichment in IgG4 indicates a deficient anti-tumor antibody response; and/or
8. extent of IgG2, wherein high levels of or enrichment in IgG2 indicates a deficient anti-tumor antibody response; and
f. optionally identifying and quantifying among the isolated antibodies any such antibodies that recognize specific tumor markers on the tumor with the properties above delineated for each marker;
wherein:
x. the isolated antibodies with increased Fc sialylation or fucosylation are subjected to antibody modification; and/or
y. the isolated antibodies are subjected to depletion of IgG4 antibodies; and/or
z. the isolated antibodies are subjected to depletion of IgG2 antibodies; and/or
aa. the isolated antibodies are subjected to enrichment of antibodies with active effector function.

23. The method of claim 22, wherein the endogenous anti-tumor antibodies weakly induce tumor cell killing in vitro and/or are ineffective at or prevent tumor elimination in vivo.

24. The method of any one of claims 22-23, wherein the endogenous anti-tumor antibodies have defective Fc effector function or suppress IgG effector function.

25. The method of any one of claims 22-24, wherein the extent of Fc sialylation is determined by Siglec-7, Siglec-3 or Sambucus nigra agglutinin (SNA) binding.

26. The method of any one of claims 22-25, wherein the extent of Fc fucosylation is determined by FcγRIIa, FcγRIIb, and/or FcγRIIIa interaction.

27. The method of any one of claims 22-26, wherein the extent of FcγRIIa, FcγRIIIa, FcγRIIb, and/or FcγRIV interaction is determined by receptor binding.

28. The method of any one of claims 22-27, wherein the extent of FcγRIIIa interaction is determined by receptor binding.

29. The method of any one of claims 22-28, wherein the tumor binding property of each antibody type and/or subtype is determined.

30. The method of any one of claims 22-29, wherein effector functional attributes are among cell killing, phagocytosis, complement response, cytokine release, and vaccination.

31. The method of any one of claims 22-30, wherein the bodily fluid is blood, plasma, serum, ascites or pleural effusion.

32. The method of any one of claims 22-31, wherein the subjected to antibody modification comprises desialylating the antibodies, such as by using a neuraminidase.

33. The method of any one of claims 22-32, wherein the subjected to antibody modification comprises defucosylating the antibodies, such as by using alpha-fucosidase.

34. The method of any one of claims 22-33, wherein enrichment of antibodies comprises increasing the ratio of IgG3 to IgG4,and/or increasing the ratio of IgG1 to IgG2, and/or increasing the ratio of IgG1 to IgG4, and/or increasing the ratio of IgG3 to IgG2, and/or increasing the ratio of the combination of IgG3 and IgG1 to IgG2, and/or increasing the ratio of the combination of IgG3 and IgG1 to IgG4, and/or increasing the ratio of IgG1 to the combination of IgG2 and IgG4, or any combination thereof.

35. The method of claim 34 wherein the isolated antibodies are enriched for IgG3 and/or IgG1.

36. A method for treating a subject having a tumor and endogenous anti-tumor antibodies comprising enhancing the anti-tumor efficacy of endogenous anti-tumor antibodies from the subject as described in any one of claims 22-35, and administering the modified, enriched and/or depleted antibodies to the subject.

37. The method of claim 36 wherein the modified anti-tumor antibodies are administered parenterally or intratumorally.

38. The method of claim 36-37, wherein the treatment shrinks or eliminates the tumor.

39. A method for treating a subject having a tumor and endogenous anti-tumor antibodies, the method comprising the steps of:
a. obtaining a sample of the tumor and/or a bodily fluid from the subject;
b. isolating antibodies bound to the tumor and/or antibodies in the bodily fluid that bind to the tumor;
c. identifying and quantifying antibody types among the isolated antibodies;
d. identifying and quantifying IgG antibody subtypes among any IgG antibodies therein; and
e. identifying among each of the antibody types and/or subtypes therein one or more Fc properties comprising from among:
1. extent of Fc sialylation, wherein increased Fc sialylation indicates defective IgG effector function or active suppression of IgG effector function;
2. extent of Fc fucosylation, wherein increased Fc fucosylation indicates defective IgG effector function; and/or
3. extent of FcγRIIa interaction, wherein decreased FcγRIIa interaction indicates defective IgG effector function; and/or
4. extent of FcγRIIIa interaction, wherein decreased FcγRIIIa interaction indicates defective IgG effector function; and/or
5. extent of FcγRIIb interaction, wherein increased FcγRIIb interaction indicates defective IgG effector function; and/or
6. extent of FcγRIV interaction, wherein decreased FcγRIV interaction indicates defective IgG effector function; and/or
7. extent of IgG4, wherein high levels of or enrichment in IgG4 indicates a deficient anti-tumor antibody response; and/or
8. extent of IgG2, wherein high levels of or enrichment in IgG2 indicates a deficient anti-tumor antibody response; and
f. optionally identifying and quantifying among the isolated antibodies any such antibodies that recognize specific tumor markers on the tumor with the properties above delineated for each marker;
wherein
x. modifying the isolated antibodies with increased Fc sialylation or fucosylation by antibody modification; and/or
y. modifying the isolated antibodies by depletion of IgG4 antibodies; and/or
z. modifying the isolated antibodies by depletion of IgG2 antibodies; and/or
aa. modifying the isolated antibodies by enrichment of antibodies with active effector function; and
administering to the subject any one or more of the modified, deleted or enriched antibodies.

40. The method of claim 39, wherein the endogenous anti-tumor antibodies weakly induce tumor cell killing in vitro and/or are ineffective at or prevent tumor elimination in vivo.

41. The method of any one of claims 39-40, wherein the endogenous anti-tumor antibodies have defective Fc effector function or suppress IgG effector function.

42. The method of any one of claims 39-41, wherein the extent of Fc sialylation is determined by Siglec-7, Siglec-3 or Sambucus nigra agglutinin (SNA) binding.

43. The method of any one of claims 39-42, wherein the extent of Fc fucosylation is determined by FcγRIIIa, FcγRIIa, FcγRIIb and/or FcγRIV interaction.

44. The method of any one of claims 39-43, wherein the extent of FcγRIIIa, FcγRIIa, FcγRIIb and/or FcγRIV interaction is determined by receptor binding.

45. The method of any one of claims 39-44, wherein the extent of FcγRIIIa interaction is determined by receptor binding.

46. The method of any one of claims 39-45, wherein the tumor binding property of each antibody type and/or subtype is determined.

47. The method of any one of claims 39-46, wherein effector functional attributes are among cell killing, phagocytosis, complement response, cytokine release, and vaccination.

48. The method of any one of claims 39-47, wherein the bodily fluid is blood, plasma, serum, ascites or pleural effusion.

49. The method of any one of claims 39-48, wherein the modifying the isolated antibodies comprises defucosylating the antibodies, such as by using alpha-fucosidase.

50. The method of any one of claims 39-49, wherein the modifying the isolated antibodies comprises desialylating the antibodies, such as by using a neuraminidase.

51. The method of any one of claims 39-50, wherein the modifying the isolated antibodies comprises enrichment of antibodies with active effector function.

52. The method of claim 51 wherein enrichment of antibodies comprises increasing the ratio of IgG3 to IgG4,and/or increasing the ratio of IgG1 to IgG2, and/or increasing the ratio of IgG1 to IgG4, and/or increasing the ratio of IgG3 to IgG2, and/or increasing the ratio of the combination of IgG3 and IgG1 to IgG2, and/or increasing the ratio of the combination of IgG3 and IgG1 to IgG4, and/or increasing the ratio of IgG1 to the combination of IgG2 and IgG4, or any combination thereof.

53. The method of claim 51 wherein the isolated antibodies are enriched for IgG3 and/or IgG1.

54. The method of claim 39 wherein the treatment shrinks or eliminates the tumor.

55. A method for treating a subject having a tumor and endogenous anti-tumor antibodies, comprising administering an enzyme that desialylates the antibodies, such as a neuraminidase, or an enzyme that defucosylates the antibodies, such as alpha-fucosidase, intratumorally or in proximity to the tumor.

56. The method of claim 55 wherein the endogenous anti-tumor antibodies have increased Fc sialylation.

57. A method for treating a subject having a tumor and endogenous anti-tumor antibodies, the method comprising the steps of:
a. obtaining a sample of the tumor and/or a bodily fluid from the subject;
b. isolating antibodies bound to the tumor and/or antibodies in the bodily fluid that bind to the tumor;
c. identifying and quantifying antibody types among the isolated antibodies;
d. identifying among each of the antibody types and/or subtypes therein one or more Fc properties comprising from among:
1. extent of Fc sialylation, wherein increased Fc sialylation indicates defective IgG effector function or active suppression of IgG effector function; and/or
2. extent of Fc fucosylation, wherein increased Fc fucosylation indicates defective IgG effector function; and/or
3. extent of FcγRIIa interaction, wherein decreased FcγRIIa interaction indicates defective IgG effector function; and/or
4. extent of FcγRIIIa interaction, wherein decreased FcγRIIIa interaction indicates defective IgG effector function;
5. extent of FcγRIIb interaction, wherein decreased FcγRIIb interaction indicates defective IgG effector function; and/or
6. extent of FcγRIV interaction, wherein decreased FcγRIV interaction indicates defective IgG effector function; and
administering an enzyme, such as a desialylase such as neuraminidase, or a defucosylase, such as alpha-fucosidase, to modify endogenous anti-tumor antibodies in situ, intratumorally or proximal to the tumor.

58. The method of any one of claims 55-57, wherein the endogenous anti-tumor antibodies weakly induce tumor cell killing in vitro and/or are ineffective at or prevent tumor elimination in vivo.

59. The method of any one of claims 55-58, wherein the endogenous anti-tumor antibodies have defective Fc effector function or suppress IgG effector function.

60. The method of claim 55-58 wherein the enzyme is administered intratumorally or peritumorally.

61. The method of any one of claims 56-60, wherein the extent of Fc sialylation is determined by Siglec-7, Siglec-3 or Sambucus nigra agglutinin (SNA) binding.

62. The method of any one of claims 56-61, wherein the extent of FcγRIIa, FcγRIIIa, FcγRIIB and/or FcγRIV interaction is determined by receptor binding.

63. The method of any one of claims 56-62, wherein the extent of FcγRIIIa, FcγRIIa, FcγRIIB and/or FcγRIV interaction is determined by receptor binding.

64. The method of any one of claims 56-63, wherein the tumor binding property of each antibody type and/or subtype is determined.

65. The method of any one of claims 56-64, wherein effector functional attributes are among cell killing, phagocytosis, complement response, cytokine release, and vaccination.

66. The method of any one of claims 56-65, wherein the bodily fluid is blood, plasma, serum, ascites or pleural effusion.

67. The method of any one of claims 55-66, wherein the treatment shrinks or eliminates the tumor.

68. The method of any one of claims 1-67, wherein the tumor is a solid tumor.

69. The method of any one of claims 1-67, wherein the tumor is high grade serous ovarian cancer.
